(19) United States
(12) Patent Application Publication
(10) Pub. No.: US 2021/0094167 A1
(43) Pub. Date: Apr. 1, 2021

(54) APPARATUS CONNECTED TO ROBOT, AND ROBOT SYSTEM INCLUDING THE ROBOT AND THE APPARATUS

(71) Applicant: LG ELECTRONICS INC., Seoul (KR)

(72) Inventors: Joonwon LEE, Seoul (KR); Reaok KO, Seoul (KR)

(73) Assignee: LG ELECTRONICS INC., Seoul (KR)

(21) Appl. No.: 16/803,155

(22) Filed: Feb. 27, 2020

(30) Foreign Application Priority Data
Sep. 27, 2019 (KR) 10-2019-0120061

(51) Int. Cl. B25J 9/00; B25J 9/16; B25J 13/02; B25J 13/08; G05D 1/00; G05D 1/02; A63B 24/00; A61B 5/0205

(52) U.S. Cl. CPC B25J 9/0003; B25J 9/163; B25J 13/025; B25J 13/08; G05D 1/0088; A61B 5/02438; A63B 24/0062; A61B 5/02055; A63B 2220/836; A63B 2220/20; G05D 1/0221

(57) ABSTRACT

An apparatus connected to a robot includes a communication interface for connecting the robot and the apparatus, a location information receiver receiving location information of the apparatus, at least one sensor including a biometric information sensor for acquiring biometric information of a user, and a processor for generating exercise information of the user based on at least one of the location information of the apparatus, the biometric information of the user acquired through the biometric sensor of the apparatus, or step count information acquired through a pedometer of the apparatus. The processor generates a control signal for controlling at least one of a moving direction or a moving speed of the robot, based on the location information of the robot or the apparatus or the information acquired through the at least one sensor, and controls the communication interface to transmit the generated control signal to the robot.

### CROSS-REFERENCE TO RELATED APPLICATIONS

The present application claims the priority benefit under 35 U.S.C. § 119 and 35 U.S.C. §365 to Korean Patent Application No. 10-2019-0120061 filed in the Republic of Korea on Sep. 27, 2019, which is hereby incorporated by reference in its entirety for all purposes as fully set forth herein.

### BACKGROUND

The present disclosure relates to an apparatus connected to a robot, and more particularly to an apparatus and a robot system connected to a robot to control the movement of the robot or supply power to the robot.

In order to manage part of factory automation, robots have been developed for industrial use. In recent years, the application fields of robots have been further expanded. Medical robots, aerospace robots, and robots that can be used in everyday life are being developed.

In particular, pet robots modeled after a shape of a pet such as a dog can provide emotional satisfaction to users. Such pet robots can operate similar to a real pet and output a sound. Since pet robots need not be fed or cleaned up, it is possible to provide the same emotional satisfaction as a real pet to busy modern people while reducing inconvenience and burden.

Meanwhile, people can feel emotional satisfaction as they spend part of their daily lives with their pets. For example, people can take health care or emotional stability by walking with their pets. A conventional pet robot has a limitation that its use is limited to simple entertainment or crime prevention. Therefore, there is a need for a method capable of expanding the spread of pet robots by providing more various functions, such as walking.

### SUMMARY

Embodiments provide an apparatus connected to a robot to provide a function for a user's health care.

Embodiments of the present invention also provide an apparatus capable of controlling the movement of the robot when moving in the outside, such as walking, or supplying power when the robot runs out of power.

In one embodiment, an apparatus connected to a robot includes: a communication interface configured to connect the robot and the apparatus; a location information receiver configured to receive location information of the apparatus; at least one sensor including a biometric information sensor configured to acquire biometric information of a user; and a processor configured to generate exercise information of the user based on at least one of the location information of the apparatus, the biometric information of the user acquired through the biometric sensor of the apparatus, or step count information acquired through a pedometer of the apparatus, wherein the processor is configured to: generate a control signal for controlling at least one of a moving direction or a moving speed of the robot, based on the location information of the robot or the apparatus or the information acquired through the at least one sensor; and control the communication interface to transmit the generated control signal to the robot.

The biometric information sensor can be configured to contact a part of a user's body to acquire the biometric information, the biometric information can include at least one of heart rate, pulse characteristics, body temperature, water content, or oxygen saturation, and the exercise information can include at least one of a moving distance, a step count, or the acquired biometric information of the user.

The processor can be configured to: detect that the location information corresponds to a location within a predetermined distance from an inaccessible area according to map information, based on the map information acquired from a memory or the communication interface and the location information of the robot or the apparatus; generate a control signal for changing the moving direction of the robot so as to be spaced apart from the inaccessible area by a predetermined distance or more; and control the communication interface to transmit the generated control signal to the robot.

The processor can be configured to generate a control signal for reducing the moving speed of the robot when a user's heart rate detected by the biometric information sensor is higher than a reference heart rate.

The at least one sensor can further include a distance sensor configured to detect a distance between the robot and the apparatus, and the processor can be configured to generate a control signal for increasing the moving speed of the robot when the detected distance is shorter than a reference distance.

The processor can be configured to generate a control signal for reducing the moving speed of the robot or changing the moving direction of the robot when the detected distance is longer than a reference distance, and control the communication interface to transmit the generated control signal to the robot.

The processor can be configured to: re-detect the distance between the robot and the apparatus after a predetermined time elapses from a time point when the control signal is transmitted; and output a notification through at least one of a display, a speaker, a light source, or a vibration motor when the re-detected distance is longer than the reference distance.

The apparatus can further include a cable connected to the robot by a wire (or other mechanism), wherein the at least one sensor can further include a tension sensor configured to detect a tension of the cable, and when a sensing value of the tension sensor is greater than a reference sensing value, the processor can be configured to generate a control signal for reducing the moving speed of the robot or changing the moving direction of the robot.

The apparatus can further include an input interface configured to receive an adjustment request for adjusting the moving speed or the moving direction of the robot, wherein the processor can be configured to generate a control signal for controlling the moving speed or the moving direction of the robot according to the received adjustment request.

The apparatus can further include a cable connected to the robot by wire and provided with a power cable, wherein the processor can be configured to: acquire battery level information of the robot through the communication interface; and perform a control such that power is supplied (from the apparatus) to a battery of the robot through the power cable, based on the acquired battery level information.

The processor can be configured to store the exercise information of the user in a memory, or control the communication interface to transmit the exercise information to a server or a mobile terminal of the user.

In one embodiment, a control method of an apparatus connected to a robot includes: detecting a connection to the robot; when an exercise mode is started, acquiring and accumulating exercise data including at least one of a location of the apparatus, biometric information of a user acquired through a biometric information sensor of the apparatus, or a step count acquired through a pedometer of the apparatus; generating a control signal for controlling at least one of a moving direction or a moving speed of the robot, based on location information of the robot or the apparatus or information acquired through at least one sensor provided in the apparatus; transmitting the generated control signal to the robot; and when the exercise mode is ended, generating exercise information of the user based on the acquired and accumulated exercise data.

In one embodiment, a robot system includes the apparatus and a robot connected to the apparatus.

The details of one or more embodiments are set forth in the accompanying drawings and the description below. Other features will be apparent from the description and drawings, and from the claims.

### BRIEF DESCRIPTION OF THE DRAWINGS

FIG. 1 illustrates an AI device including a robot according to an embodiment.

FIG. 2 illustrates an AI server connected to a robot according to an embodiment.

FIG. 3 illustrates an AI system including the robot according to an embodiment.

FIG. 4 is a block diagram illustrating a control configuration of the robot according to an embodiment.

FIG. 5 is a perspective view of the robot according to an embodiment.

FIG. 6 is a perspective view of an apparatus connected to the robot illustrated in FIG. 5 according to an embodiment.

FIG. 7 is a block diagram illustrating a control configuration of the apparatus illustrated in FIG. 6.

FIG. 8 is a flowchart for describing a control operation of an apparatus connected to a robot according to an embodiment.

FIG. 9 illustrates an example of a user walking with the robot using the apparatus connected to the robot according to an embodiment.

FIG. 10 is a flowchart for describing an operation in which the apparatus according to the embodiment controls movement characteristics of the robot.

FIG. 11 is a flowchart for explaining an example of a control operation performed by the apparatus according to the embodiment, based on a distance to the robot.

FIG. 12 illustrates an example in which exercise information acquired by the apparatus according to the embodiment is provided through a user terminal.

FIG. 13 is a flowchart for describing an operation in which the apparatus according to the embodiment supplies power to the robot based on a battery state of the robot.

FIG. 14 illustrates an example relating to the operation of the apparatus illustrated in FIG. 13.

### DETAILED DESCRIPTION OF THE EMBODIMENTS

Hereinafter, embodiments disclosed herein will be described in detail with reference to the accompanying drawings. The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings.

A robot can refer to a machine that automatically processes or operates a given task by its own ability. In particular, a robot having a function of recognizing an environment and performing a self-determination operation can be referred to as an intelligent robot.

Robots can be classified into industrial robots, medical robots, home robots, military robots, and the like according to the use, purpose or field.

The robot includes a driver can include an actuator or a motor and can perform various physical operations, such as moving a robot joint. In addition, a movable robot can include a wheel, a brake, a propeller, and the like in a driver, and can travel on the ground through the driver or fly in the air.

Artificial intelligence refers to the field of studying artificial intelligence or methodology for making artificial intelligence, and machine learning refers to the field of defining various issues dealt with in the field of artificial intelligence and studying methodology for solving the various issues, as known in the related art. Machine learning is defined as an algorithm that enhances the performance of a certain task through a steady experience with the certain task, as known in the related art.

An artificial neural network (ANN) is a model used in machine learning and can mean a whole model of problem-solving ability which is composed of artificial neurons (nodes) that form a network by synaptic connections. The artificial neural network can be defined by a connection pattern between neurons in different layers, a learning process for updating model parameters, and an activation function for generating an output value.

The artificial neural network can include an input layer, an output layer, and optionally one or more hidden layers. Each layer includes one or more neurons, and the artificial neural network can include a synapse that links neurons to neurons. In the artificial neural network, each neuron can output the function value of the activation function for input signals, weights, and deflections input through the synapse.

Model parameters refer to parameters determined through learning and include a weight value of synaptic connection and deflection of neurons. A hyperparameter means a parameter to be set in the machine learning algorithm before learning, and includes a learning rate, a repetition number, a mini batch size, and an initialization function.

The purpose of the learning of the artificial neural network can be to determine the model parameters that minimize a loss function. The loss function can be used as an index to determine optimal model parameters in the learning process of the artificial neural network.

Machine learning can be classified into supervised learning, unsupervised learning, and reinforcement learning according to a learning method.

The supervised learning can refer to a method of learning an artificial neural network in a state in which a label for learning data is given, and the label can mean the correct answer (or result value) that the artificial neural network must infer when the learning data is input to the artificial neural network. The unsupervised learning can refer to a method of learning an artificial neural network in a state in which a label for learning data is not given. The reinforcement learning can refer to a learning method in which an agent defined in a certain environment learns to select a behavior or a behavior sequence that maximizes cumulative compensation in each state.

Machine learning, which is implemented as a deep neural network (DNN) including a plurality of hidden layers among artificial neural networks, is also referred to as deep learning, and the deep learning is part of machine learning. In the following, machine learning is used to mean deep learning.

Self-driving refers to a technique of driving for oneself, and a self-driving vehicle refers to a vehicle that travels without an operation of a user or with a minimum operation of a user.

For example, the self-driving can include a technology for maintaining a lane while driving, a technology for automatically adjusting a speed, such as adaptive cruise control, a technique for automatically traveling along a predetermined route, and a technology for automatically setting and traveling a route when a destination is set.

The vehicle can include a vehicle having only an internal combustion engine, a hybrid vehicle having an internal combustion engine and an electric motor together, and an electric vehicle having only an electric motor, and can include not only an automobile, but also a train, a motorcycle, and the like.

At this time, the self-driving vehicle can be regarded as a robot having a self-driving function.

FIG. 1 illustrates an AI device 100 including a robot according to an embodiment of the present invention. All components of various devices and systems including an AI device, a robot, a server, etc. according to all embodiments of the present invention are operatively coupled and configured.

The AI device 100 can be implemented by a stationary device or a mobile device, such as a TV, a projector, a mobile phone, a smartphone, a desktop computer, a notebook, a digital broadcasting terminal, a personal digital assistant (PDA), a portable multimedia player (PMP), a navigation device, a tablet PC, a wearable device, a set-top box (STB), a DMB receiver, a radio, a washing machine, a refrigerator, a desktop computer, a digital signage, a robot, a vehicle, and the like.

Referring to FIG. 1, the AI device 100 can include a communication interface 110, an input interface 120, a learning processor 130, a sensor 140, an output interface 150, a memory 170, and a processor 180.

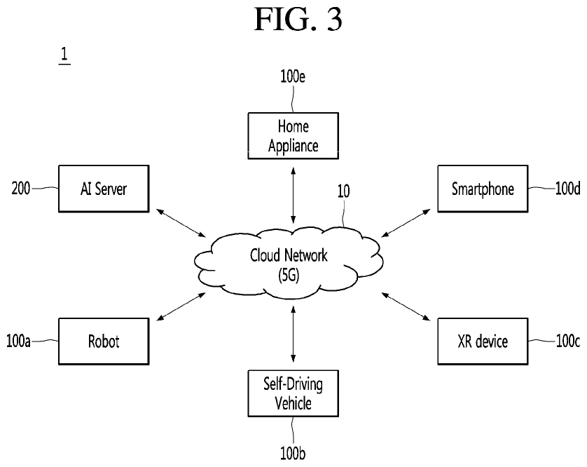

The communication interface 110 can transmit and receive data to and from external devices, such as other AI devices 100a to 100e and the AI server 200 (see FIG. 3) by using wired/wireless communication technology. For example, the communication interface 110 can transmit and receive sensor information, a user input, a learning model, and a control signal to and from external devices.

The communication technology used by the communication interface 110 includes GSM (Global System for Mobile communication), CDMA (Code Division Multi Access), LTE (Long Term Evolution), 5G, WLAN (Wireless LAN), Wi-Fi (Wireless-Fidelity), Bluetooth™, RFID (Radio Frequency Identification), Infrared Data Association (IrDA), ZigBee™, NFC (Near Field Communication), and the like.

The input interface 120 can acquire various kinds of data.

At this time, the input interface 120 can include a camera for inputting a video signal, a microphone for receiving an audio signal, and a user input interface for receiving information from a user. The camera or the microphone can be treated as a sensor, and the signal acquired from the camera or the microphone can be referred to as sensing data or sensor information.

The input interface 120 can acquire a learning data for model learning and an input data to be used when an output is acquired by using learning model. The input interface 120 can acquire raw input data. In this case, the processor 180 or the learning processor 130 can extract an input feature by preprocessing the input data.

The learning processor 130 can learn a model composed of an artificial neural network by using learning data. The learned artificial neural network can be referred to as a learning model. The learning model can be used to an infer result value for new input data rather than learning data, and the inferred value can be used as a basis for determination to perform a certain operation.

At this time, the learning processor 130 of the AI device 100 can perform AI processing together with the learning processor 240 of the AI server 200.

At this time, the learning processor 130 of the AI device 100 can include a memory integrated or implemented in the AI device 100. Alternatively, the learning processor 130 can be implemented by using the memory 170, an external memory directly connected to the AI device 100, or a memory held in an external device.

The sensor 140 can acquire at least one of internal information about the AI device 100, ambient environment information about the AI device 100, and user information by using various sensors.

Examples of the various sensors included in the sensor 140 can include a proximity sensor, an illuminance sensor, an acceleration sensor, a magnetic sensor, a gyro sensor, an inertial sensor, an RGB sensor, an IR sensor, a fingerprint recognition sensor, an ultrasonic sensor, an optical sensor, a microphone, a lidar, and a radar.

The output interface 150 can generate an output related to a visual sense, an auditory sense, or a haptic sense.

At this time, the output interface 150 can include a display for outputting time information, a speaker for outputting auditory information, and a haptic module for outputting haptic information.

The memory 170 can store data that supports various functions of the AI device 100. For example, the memory 170 can store input data acquired by the input interface 120, learning data, a learning model, a learning history, and the like.

The processor 180 can determine at least one executable operation of the AI device 100 based on information determined or generated by using a data analysis algorithm or a machine learning algorithm. The processor 180 can control the components of the AI device 100 to execute the determined operation.

To this end, the processor 180 can request, search, receive, or utilize data of the learning processor 130 or the memory 170. The processor 180 can control the components of the AI device 100 to execute the predicted operation or the operation determined to be desirable among the at least one executable operation.

When the connection of an external device is required to perform the determined operation, the processor 180 can generate a control signal for controlling the external device and can transmit the generated control signal to the external device.

The processor 180 can acquire intention information for the user input and can determine the user's requirements based on the acquired intention information.

The processor 180 can acquire the intention information corresponding to the user input by using at least one of a speech to text (STT) engine for converting speech input into a text string or a natural language processing (NLP) engine for acquiring intention information of a natural language.

At least one of the STT engine or the NLP engine can be configured as an artificial neural network, at least part of which is learned according to the machine learning algorithm. At least one of the STT engine or the NLP engine can be learned by the learning processor 130, can be learned by the learning processor 240 of the AI server 200, or can be learned by their distributed processing.

The processor 180 can collect history information including the operation contents of the AI apparatus 100 or the user's feedback on the operation and can store the collected history information in the memory 170 or the learning processor 130 or transmit the collected history information to the external device such as the AI server 200. The collected history information can be used to update the learning model.

The processor 180 can control at least part of the components of AI device 100 so as to drive an application program stored in memory 170. Furthermore, the processor 180 can operate two or more of the components included in the AI device 100 in combination so as to drive the application program.

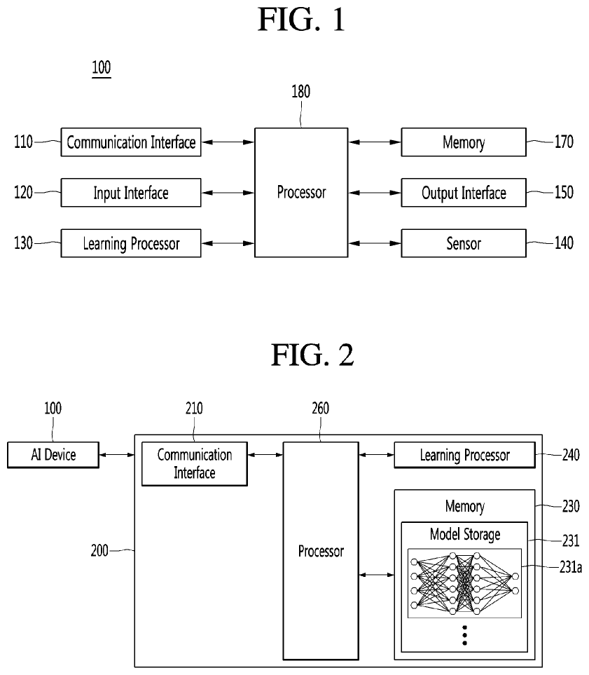

FIG. 2 illustrates an AI server 200 connected to a robot according to an embodiment of the present invention.

Referring to FIG. 2, the AI server 200 can refer to a device that learns an artificial neural network by using a machine learning algorithm or uses a learned artificial neural network. The AI server 200 can include a plurality of servers to perform distributed processing, or can be defined as a 5G network. At this time, the AI server 200 can be included as a partial configuration of the AI device 100, and can perform at least part of the AI processing together.

The AI server 200 can include a communication interface 210, a memory 230, a learning processor 240, a processor 260, and the like.

The communication interface 210 can transmit and receive data to and from an external device, such as the AI device 100.

The memory 230 can include a model storage 231. The model storage 231 can store a learning or learned model (or an artificial neural network 231a) through the learning processor 240.

The learning processor 240 can learn the artificial neural network 231a by using the learning data. The learning model can be used in a state of being mounted on the AI server 200 of the artificial neural network, or can be used in a state of being mounted on an external device such as the AI device 100.

The learning model can be implemented in hardware, software, or a combination of hardware and software. If all or part of the learning models are implemented in software, one or more instructions that constitute the learning model can be stored in memory 230.

The processor 260 can infer the result value for new input data by using the learning model and can generate a response or a control command based on the inferred result value.

FIG. 3 illustrates an AI system 1 according to an embodiment of the present invention.

Referring to FIG. 3, in the AI system 1, at least one of an AI server 200, a robot 100a, a self-driving vehicle 100b, an XR device 100c, a smartphone 100d, or a home appliance 100e is connected to a cloud network 10. The robot 100a, the self-driving vehicle 100b, the XR device 100c, the smartphone 100d, or the home appliance 100e, to which the AI technology is applied, can be referred to as AI devices 100a to 100e.

The cloud network 10 can refer to a network that forms part of a cloud computing infrastructure or exists in a cloud computing infrastructure. The cloud network 10 can be configured by using a 3G network, a 4G or LTE network, a 5G network or any other type of network.

That is, the devices 100a to 100e and 200 configuring the AI system 1 can be connected to each other through the cloud network 10. In particular, each of the devices 100a to 100e and 200 can communicate with each other through a base station, but can directly communicate with each other without using a base station.

The AI server 200 can include a server that performs AI processing and a server that performs operations on big data.

The AI server 200 can be connected to at least one of the AI devices constituting the AI system 1, that is, the robot 100a, the self-driving vehicle 100b, the XR device 100c, the smartphone 100d, or the home appliance 100e through the cloud network 10, and can assist at least part of AI processing of the connected AI devices 100a to 100e.

At this time, the AI server 200 can learn the artificial neural network according to the machine learning algorithm instead of the AI devices 100a to 100e, and can directly store the learning model or transmit the learning model to the AI devices 100a to 100e.

At this time, the AI server 200 can receive input data from the AI devices 100a to 100e, can infer the result value for the received input data by using the learning model, can generate a response or a control command based on the inferred result value, and can transmit the response or the control command to the AI devices 100a to 100e.

Alternatively, the AI devices 100a to 100e can infer the result value for the input data by directly using the learning model, and can generate the response or the control command based on the inference result.

Hereinafter, various embodiments of the AI devices 100a to 100e to which the above-described technology is applied will be described. The AI devices 100a to 100e illustrated in FIG. 3 can be regarded as a specific embodiment of the AI device 100 illustrated in FIG. 1.

The robot 100a, to which the AI technology is applied, can be implemented as a guide robot, a carrying robot, a cleaning robot, a wearable robot, an entertainment robot, a pet robot, an unmanned flying robot, or the like.

The robot 100a can include a robot control module for controlling the operation, and the robot control module can refer to a software module or a chip implementing the software module by hardware.

The robot 100a can acquire state information about the robot 100a by using sensor information acquired from various kinds of sensors, can detect (recognize) surrounding environment and objects, can generate map data, can determine the route and the travel plan, can determine the response to user interaction, or can determine the operation.

The robot 100a can use the sensor information acquired from at least one sensor among the lidar, the radar, and the camera so as to determine the travel route and the travel plan.

The robot 100a can perform the above-described operations by using the learning model composed of at least one artificial neural network. For example, the robot 100a can recognize the surrounding environment and the objects by using the learning model, and can determine the operation by using the recognized surrounding information or object information. The learning model can be learned directly from the robot 100a or can be learned from an external device, such as the AI server 200.

At this time, the robot 100a can perform the operation by generating the result by directly using the learning model, but the sensor information can be transmitted to the external device such as the AI server 200 and the generated result can be received to perform the operation.

The robot 100a can use at least one of the map data, the object information detected from the sensor information, or the object information acquired from the external apparatus to determine the travel route and the travel plan, and can control the driver such that the robot 100a travels along the determined travel route and travel plan.

The map data can include object identification information about various objects arranged in the space in which the robot 100a moves. For example, the map data can include object identification information about fixed objects, such as walls and doors and movable objects, such as chairs and desks. The object identification information can include a name, a type, a distance, and a position.

In addition, the robot 100a can perform the operation or can travel by controlling the driver based on the control/interaction of the user. At this time, the robot 100a can acquire the intention information of the interaction due to the user's operation or speech utterance, and can determine the response based on the acquired intention information, and can perform the operation.

The robot 100a, to which the AI technology and the self-driving technology are applied, can be implemented as a guide robot, a carrying robot, a cleaning robot, a wearable robot, an entertainment robot, a pet robot, an unmanned flying robot, or the like.

The robot 100a, to which the AI technology and the self-driving technology are applied, can refer to the robot itself having the self-driving function or the robot 100a interacting with the self-driving vehicle 100b.

The robot 100a having the self-driving function can collectively refer to a device that moves for itself along the given movement line without the user's control or moves for itself by determining the movement line by itself

The robot 100a and the self-driving vehicle 100b having the self-driving function can use a common sensing method so as to determine at least one of the travel route or the travel plan. For example, the robot 100a and the self-driving vehicle 100b having the self-driving function can determine at least one of the travel route or the travel plan by using the information sensed through the lidar, the radar, and/or the camera.

The robot 100a that interacts with the self-driving vehicle 100b exists separately from the self-driving vehicle 100b and can perform operations interworking with the self-driving function of the self-driving vehicle 100b or interworking with the user who rides on the self-driving vehicle 100b.

At this time, the robot 100a interacting with the self-driving vehicle 100b can control or assist the self-driving function of the self-driving vehicle 100b by acquiring sensor information on behalf of the self-driving vehicle 100b and providing the sensor information to the self-driving vehicle 100b, or by acquiring sensor information, generating environment information or object information, and providing the information to the self-driving vehicle 100b.

Alternatively, the robot 100a interacting with the self-driving vehicle 100b can monitor the user boarding the self-driving vehicle 100b, or can control the function of the self-driving vehicle 100b through the interaction with the user. For example, when it is determined that the driver is in a drowsy state, the robot 100a can activate the self-driving function of the self-driving vehicle 100b or assist the control of the driver of the self-driving vehicle 100b. The function of the self-driving vehicle 100b controlled by the robot 100a can include not only the self-driving function but also the function provided by the navigation system or the audio system provided in the self-driving vehicle 100b.

Alternatively, the robot 100a that interacts with the self-driving vehicle 100b can provide information or assist the function to the self-driving vehicle 100b outside the self-driving vehicle 100b. For example, the robot 100a can provide traffic information including signal information and the like, such as a smart signal, to the self-driving vehicle 100b, and automatically connect an electric charger to a charging port by interacting with the self-driving vehicle 100b like an automatic electric charger of an electric vehicle.

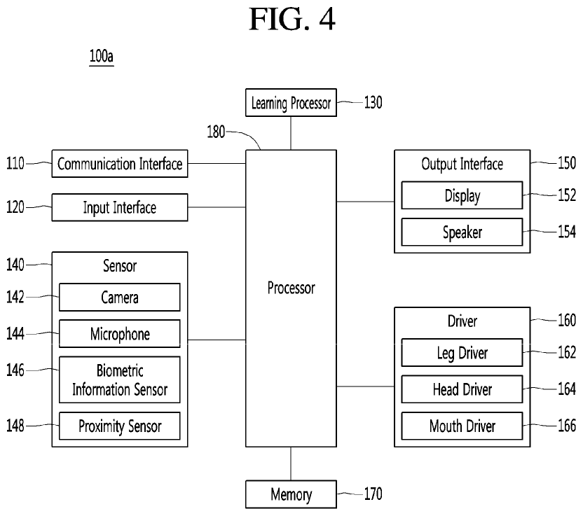

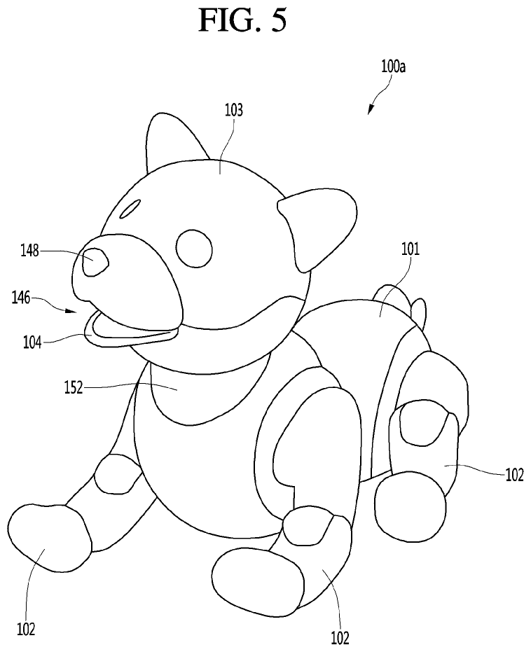

FIG. 4 is a block diagram illustrating the control configuration of the robot according to an embodiment

Referring to FIG. 4, the robot 100a can include a communication interface 110, an input interface 120, a learning processor 130, a sensor 140, an output interface 150, a driver 160, a memory 170, and a processor 180. The configurations illustrated in FIG. 4 are examples for convenience of explanation, and the robot 100a can include more or fewer configurations than those illustrated in FIG. 4.

Meanwhile, the contents related to the AI device 100 of FIG. 1 is also similarly applied to the robot 100a of the present disclosure, and redundant contents thereof will be omitted.

The communication interface 110 can include communication modules for connecting the robot 100a to a server, a mobile terminal, another robot, or the like via a network. Each of the communication modules can support any of the communication technologies described above in FIG. 1 or any other known communication technology.

For example, the robot 100a can be connected to the network via an access point, such as a router. Therefore, the robot 100a can provide a variety of information acquired through the input interface 120, the sensor 140, and the like to the server or the mobile terminal via the network. In addition, the robot 100a can receive information, data, commands, and the like from the server or the mobile terminal.

The input interface 120 can include at least one input devices for acquiring various kinds of data. For example, the at least one input device can include a physical input device, such as a button or a dial, a touch input device, such as a touch pad or a touch panel, and a microphone for receiving a voice of the user or a sound from around the robot 100a. The user can input various requests or commands through the input interface 120 to the robot 100a.

The sensor 140 can include at least one sensor for sensing a variety of information around the robot 100a.

For example, the sensor 140 can include a camera 142 for acquiring an image around the robot 100a and a microphone 144 for acquiring a voice around the robot 100a.

In addition, the sensor 140 can further include a biometric information sensor 146 for acquiring biometric information of the user.

The biometric information sensor 146 can include at least one sensor for acquiring a biometric signal related to a variety of biometric information, such as a user's heart rate, pulse characteristics (regularity, intensity, etc.), body temperature, stress, and oxygen saturation, but is not limited thereto and may include additional biometric information. For example, the biometric information sensor 146 can include various types of sensors for acquiring a biometric signal based on photoplethysmography.

The processor 180 can acquire the biometric information from the biometric signal acquired through the biometric information sensor 146. In addition, the processor 180 can acquire health state information of the user based on the acquired biometric information. According to an embodiment, the processor 180 can transmit the acquired biometric information (or biometric signal) to the server through the communication interface 110 and acquire the health state information from the server.

According to an embodiment, the sensor 140 can further include a proximity sensor 148 for detecting whether a part of the user's body is in proximity to the robot (e.g., the proximity sensor 148 detects objects around the sensor, including objects proximate/close to the sensor). In the present embodiment, the biometric information sensor 146 can be provided in a hidden state (e.g., not visible or partially not visible from outside of the robot) at a part of the robot 100a and can be exposed to the outside as the proximity of the part of the body is detected by the proximity sensor 148.

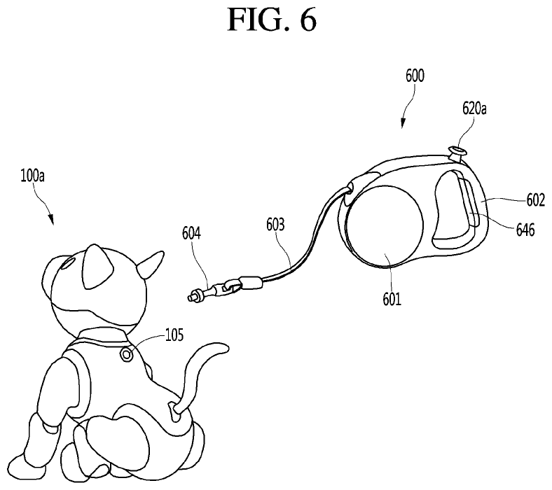

An embodiment related to the arrangement of the biometric information sensor 146 and the proximity sensor 148 will be described below with reference to FIGS. 5 and 6.

According to an embodiment, the sensor 140 can include various sensors, such as an illumination sensor, for detecting the brightness of the space in which the robot 100a is disposed, and a gyro sensor for detecting the rotation angle or tilt of the robot 100a.

The output interface 150 can output a variety of information or contents about the operation or state of the robot 100a and various services, programs, and applications executed by the robot 100a. For example, the output interface 150 can include a display 152 and a speaker 154.

The display 152 can output the variety of above-described information, messages, or contents in a graphic form. According to an embodiment, the display 152 can be implemented as a touch screen together with a touch input interface.

The speaker 154 can output the variety of above-described information, messages, or contents in a voice or sound form.

The driver 160 can include at least one configuration related to the movement of the robot 100a and the movement (rotation, tilting, etc.) of predetermined parts of the robot 100a.

For example, the driver 160 can include a leg driver 162, a head driver 164, and a mouth driver 166. Each of the drivers 162, 164, and 166 can include at least one motor for the movement or activity.

The driver 160 can include a moving device having at least one motor for the movement of (traveling, etc.) of the robot 100a. In the present disclosure, the leg driver 162 is illustrated as an example of the moving device, but when the robot 100a includes a moving structure (wheel, etc.) instead of a leg portion 102, the driver can include a moving device of a type other than the leg driver 162.

The leg driver 162 enables the movement of the robot 100a by providing a driving force for rotating at least one joint formed in the leg portion 102 (see FIG. 5) of the robot 100a.

The head driver 164 corresponds to a configuration for rotating or tilting a head portion 103 (see FIG. 5) of the robot 100a.

The mouth driver 166 corresponds to a configuration for opening or closing a mouth portion 104 of the robot 100a. As will be described below with reference to FIGS. 5 and 6, the mouth driver 166 rotates a rotating portion (corresponding to a lower jaw) of the mouth portion 104 upward or downward, thereby enabling the opening or closing of the mouth portion 104.

The memory 170 can store various data such as control data for controlling the operations of the components included in the robot 100a and data for performing the operation based on the pressure acquired through the input interface 120 or the information acquired through the sensor 140.

In addition, the memory 170 can store program data, such as software modules or applications executed by at least one processor or controller included in the processor 180.

The memory 170 can include various storage devices, such as ROM, RAM, EPROM, flash drive, or hard drive in hardware.

The processor 180 can include at least one processor or controller for controlling the operation of the robot 100a. Specifically, the processor 180 can include at least one of a CPU, an application processor (AP), a microcomputer (or microcomputer), an integrated circuit, or an application specific integrated circuit (ASIC).

FIG. 5 is a perspective view of the robot according to an embodiment.

Referring to FIG. 5, the robot 100a according to the embodiment can be implemented as a robot having a pet shape. Although the dog-shaped robot 100a is exemplarily illustrated in FIG. 5, the shape of the robot 100a is not limited thereto, and may have a shape of any type of animal or object.

For example, the robot 100a can include a body portion 101, a leg portion 102, and a head portion 103, but the type or number of the configurations can be variously changed according to the shape of the robot 100a.

The body portion 101 can correspond to the body of the pet. For example, the body portion 101 can include components for driving the robot 100a, for example, at least one printed circuit board (PCB) on which at least some of the control components illustrated in FIG. 4 are mounted, a battery for providing power, and the like. According to an embodiment, the display 152 can be implemented in the form of a neckband detachable to the body portion 101 of the robot 100a, but is not necessarily limited thereto.

The leg portion 102 is a configuration corresponding to the leg of the pet, and is connected to the body portion 101 to enable the movement of the robot 100a.

For example, the leg portion 102 can include a plurality of legs, each of which can include configurations corresponding to the legs, feet, and joints connected thereto, respectively. The leg driver 162 described above with reference to FIG. 4 includes at least one motor for rotating the configuration corresponding to the joint, and the robot 100a can move or travel according to the driving of the leg driver 162.

The head portion 103 is a configuration corresponding to the head of the pet, and can be connected to the front or upper side of the body portion 101. The head driver 164 described above with reference to FIG. 4 includes at least one motor for rotating or tilting the head portion 103, and the head portion 103 can move according to the driving of the head driver 164.

Meanwhile, the head portion 103 can include at least some of components included in the sensor 140, such as the camera 142, the biometric information sensor 146, and the proximity sensor 148. For example, the camera 142 can be disposed at a position corresponding to the eye of the pet, but is not necessarily limited thereto.

The mouth portion 104 corresponding to the mouth of the pet can be formed on one side of the head portion 103. For example, the mouth portion 104 can include a fixing portion (for example, the upper jaw of the pet) formed in the head portion 103, and a rotating portion (for example, the lower jaw of the pet) disposed below the fixing portion and rotatable up and down.

The mouth driver 166 can include a motor for opening or closing the mouth portion 104 (for example, rotating the rotating portion in a vertical direction). In detail, the mouth driver 166 can be provided inside the head portion 103 and can be connected to the rotating portion of the mouth portion 104. As the mouth driver 166 is driven, the rotating portion can rotate upward or downward. The mouth portion 104 can be closed when the rotating portion rotates upward, and the mouth portion 104 can be opened when the rotating portion rotates downward.

The biometric information sensor 146 can be provided inside the mouth portion 104. For example, the biometric information sensor 146 can be disposed at a position corresponding to the upper side of the rotating portion or the tongue of the pet. Accordingly, the biometric information sensor 146 not be exposed to the outside when the mouth portion 104 is closed, thereby minimizing the risk of contamination or damage due to external factors.

Meanwhile, the proximity sensor 148 can be provided at a position corresponding to the nose of the robot 100a. The proximity sensor 148 can be implemented as various types of sensors capable of detecting a distance to an object, such as an infrared sensor.

According to an embodiment, the proximity sensor 148 can detect that a part of the user's body is proximity to the mouth portion 104. For example, when the user health monitoring function is executed, the processor 180 can drive the mouth driver 166 to open the mouth portion 104 based on the detection result of the proximity sensor 148. As the mouth portion 104 is opened, the part of the user's body (for example, a finger) comes into contact with the biometric information sensor 146, and the processor 180 can acquire the biometric information of the user through the biometric information sensor 146.

FIG. 6 is a perspective view of an apparatus connected to the robot illustrated in FIG. 5 according to an embodiment.

Referring to FIG. 6, an apparatus 600 is wired to the robot 100a to check the state (power, remaining battery level, error, etc.) of the robot 100a, control the movement of the robot 100a, or supply power to the battery of the robot 100a.

The apparatus 600 can include a body 601, a handle portion 602, and robot connection portions 603 and 604.

The body 601 can define the overall appearance of the apparatus 600. Inside the body 601, various control configurations related to the operation of the apparatus 600, one or more batteries, and a part of the robot connection portion 603 can be accommodated.

The handle portion 602 can be formed at one side of the body 601. For example, the handle portion 602 can have a form that is easy to be gripped by a user. A part of the handle portion 602 can be formed with a biometric information sensor 646 for contacting the user's hand to acquire biometric information of the user.

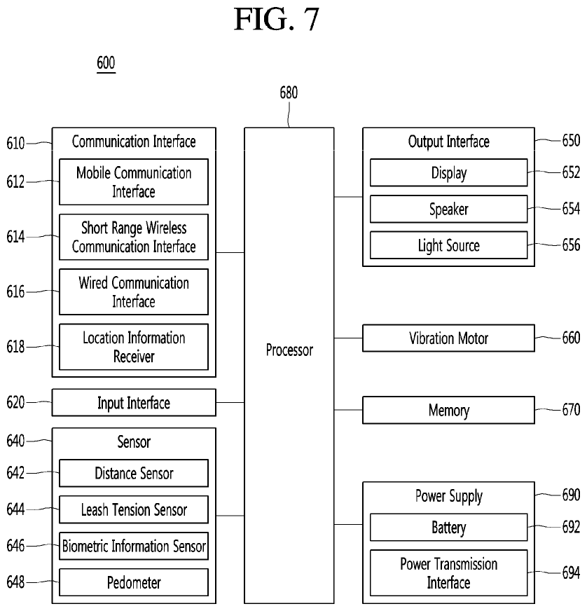

According to an embodiment, an input interface 620 (see FIG. 7) can be formed at an upper portion of the handle portion 602 or a position adjacent to the handle portion 602. Accordingly, the user can conveniently manipulate the input interface 620 through a finger while holding the handle portion 602. For example, the input interface 620 can include various input devices, such as a joystick 620a, a button, a dial, or a lever or the like.

The robot connection portions 603 and 604 can include a cable 603 and a terminal 604 for wired connection between the apparatus 600 and the robot 100a.

The cable 603 can be accommodated in the body 601 when the apparatus 600 is not in use, and at least a part of the cable 603 can be drawn out when the apparatus 600 is in use. In addition, the length of the portion of the cable 603 drawn out to the outside can increase or decrease according to the change in the distance between the robot 100a and the apparatus 600. That is, the cable 603 can function as a kind of a leash for the robot 100a.

A communication cable for wired communication between the apparatus 600 and the robot 100a, and a power cable for power transmission between the apparatus 600 and the robot 100a can be provided inside the cable 603. The communication cable can be connected to a wired communication interface 616 (see FIG. 7) provided in the apparatus 600, and the power cable can be connected to a battery 692 (see FIG. 7) and/or a power transmission interface 694 (see FIG. 7) provided in the apparatus 600.

Meanwhile, a leash tension sensor 644 directly or indirectly connected to one end of the cable 603 can be provided in the body 601. The leash tension sensor 644 can include various types of sensors for measuring the tension of the cable 603 and the torque of an axis (not shown) around which the cable 603 in the body 601 is wound. The apparatus 600 can control the movement characteristics (a moving speed, a moving acceleration or a moving direction) of the robot 100a, such that the distance between the robot 100a and the apparatus 600 does not exceed the maximum distance, based on the sensing value of the leash tension sensor 644.

The terminal 604 can be formed at the other end of the cable 603. The terminal 604 can be inserted into a terminal hole 105 formed at a predetermined position of the robot 100a to connect (e.g., physically connect and electrically connect) the robot 100a and the apparatus 600.

FIG. 7 is a block diagram illustrating the control configuration of the apparatus illustrated in FIG. 6.

Referring to FIG. 7, the apparatus 600 can include a communication interface 610, an input interface 620, a sensor 640, an output interface 650, a memory 670, a processor 680, and a power supply 690. The control configuration of the apparatus 600 according to the embodiment is not limited to the example of FIG. 7, and the apparatus 600 can include more or fewer configurations.

Meanwhile, the contents related to the AI device 100 of FIG. 1 is also similarly applied to the apparatus 600, and redundant contents thereof will be omitted.

The communication interface 610 can include at least one communication interface for connecting the apparatus 600 to a robot 100a, a mobile terminal, a server, and the like. The at least one communication interface can support any of the communication technologies described above in FIG. 1. The at least one communication interface can be implemented as a modem, a transceiver, or the like.

For example, the communication interface 110 can include a mobile communication interface 612, a short range wireless communication interface 614, a wired communication interface 616, and a location information receiver 618. The apparatus 600 can be connected to the server, the mobile terminal, and/or the robot 100a through the mobile communication interface 612, and can be connected to the mobile terminal and/or the robot 100a through the short range wireless communication interface 614. In addition, the apparatus 600 can be connected to the robot 100a through the wired communication interface 616. The apparatus 600 can receive the location information of the apparatus 600 from a location information provider (GPS satellite, etc.) through the location information receiver 618.

The input interface 620 can include at least one input interface for acquiring various types of data from the user or the like. For example, the at least one input interface can include a physical input device, such as a button or a dial, a touch input device, such as a touch pad or a touch panel, and a microphone for receiving a voice of the user or a sound around the apparatus 600. The user can input various requests or commands through the input interface 620 to the apparatus 600.

The sensor 640 can include at least one sensor for measuring the distance between the robot 100a and the apparatus 600 or acquiring data related to the exercise information of the user.

For example, the sensor 640 can include a distance sensor 642 for measuring the distance between the robot 100a and the apparatus 600. The distance sensor 642 can include various sensors, such as an ultrasonic sensor, a laser sensor, a proximity sensor, and/or a camera, which is capable of measuring or estimating the distance between the robot 100a and the apparatus 600.

The sensor 640 can include a leash tension sensor 644 for detecting whether the distance between the robot 100a and the apparatus 600 exceeds the maximum distance. The leash tension sensor 644 has been described above with reference to FIG. 6.

In addition, the sensor 640 can include a biometric information sensor 646 for acquiring biometric information and a pedometer 648 for measuring a step count in relation to the user's exercise information (e.g., for measuring a step count of the user). The biometric information can include a variety of information, such as heart rate, pulse characteristics (regularity, intensity, etc.), body temperature, water content, or oxygen saturation. For example, the biometric information sensor 646 can include various types of sensors for acquiring biometric information according to a method, such as application of microcurrent or photoplethysmography.

The processor 680 can generate exercise information of the user based on the biometric information acquired through the biometric information sensor 646 and the step count information acquired through the pedometer 648. The exercise information can be output through the output interface 650 of the apparatus 600 or the output interface 150 of the robot 100a, or can be transmitted to the server or the mobile terminal through the communication interface 610.

According to an embodiment, the sensor 640 can further include a gyro sensor for detecting a rotation angle or a tilt of the apparatus 600. For example, the user can rotate or tilt the apparatus 600 so as to control the moving direction or the moving speed of the robot 100a. The processor 680 can measure the degree of rotation or tilt of the apparatus 600 through the gyro sensor, generate a control signal for adjusting the moving direction or the moving speed of the robot 100a based on the measurement result, and transmit the generated control signal to the robot 100a.

The output interface 650 can output a variety of information, such as an operation or state of the apparatus 600 or the robot 100a, and exercise information of the user. For example, the output interface 650 can include a display 652, a speaker 654, a light source 656, and the like.

The display 652 can output the variety of above-described information, messages, or contents in a graphic form. According to an embodiment, the display 652 can be implemented as a touch screen together with a touch input interface.

The speaker 654 can output the variety of above-described information, messages, or contents in a voice or sound form. The light source 656 can notify the user of an occurrence of an event by outputting light of a color or a pattern corresponding to a specific event occurring in the apparatus 600 or the robot 100a.

According to an embodiment, the apparatus 600 can include a vibration motor 660 for vibrating the apparatus 600 or the handle portion 602. For example, the processor 680 can vibrate the apparatus 600 by driving the vibration motor 660 when a specific event (battery shortage, theft, etc.) occurs in the apparatus 600 or the robot 100a. Since the user is holding the apparatus 600, the user can easily detect the vibration of the apparatus 600 and can quickly recognize that an event occurs in the apparatus 600 or the robot 100a according to the detected vibration.

The memory 670 can store various data such as control data for controlling the operations of the components included in the apparatus 600 and data for performing the operation based on the pressure acquired through the input interface 620 or the information acquired through the sensor 640.

In addition, the memory 670 can store program data, such as software modules or applications executed by at least one processor or a controller included in the processor 680.

The memory 670 can include various storage devices, such as ROM, RAM, EPROM, flash drive, or hard drive in hardware.

The processor 680 can include at least one processor or controller for controlling the operation of the apparatus 600. Specifically, the processor 680 can include at least one of a CPU, an application processor (AP), a microcomputer (or microcomputer), an integrated circuit (IC), or an application specific integrated circuit (ASIC).

The power supply 690 can include a battery 692 for supplying power necessary for driving the apparatus 600. In addition, the power supply 690 can include a power transmission interface 694 for transmitting power to the robot 100a based on the remaining battery level of the robot 100a connected to the apparatus 600.

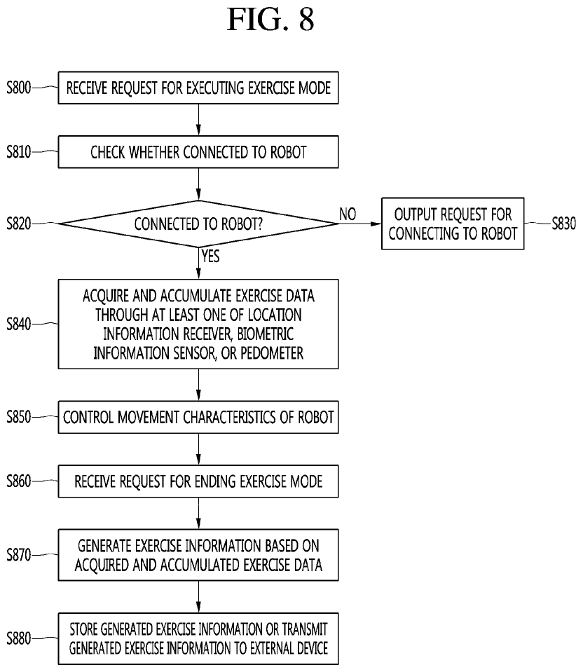

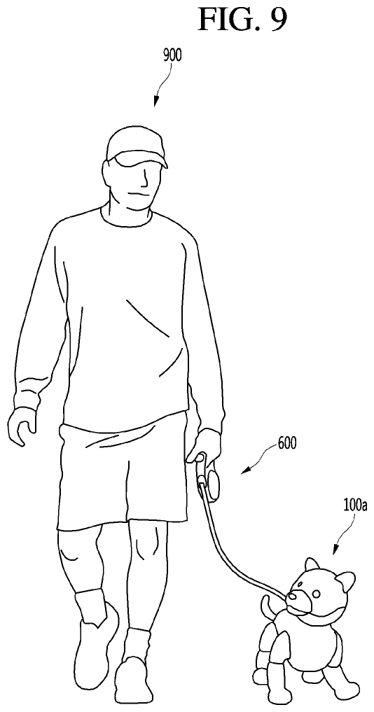

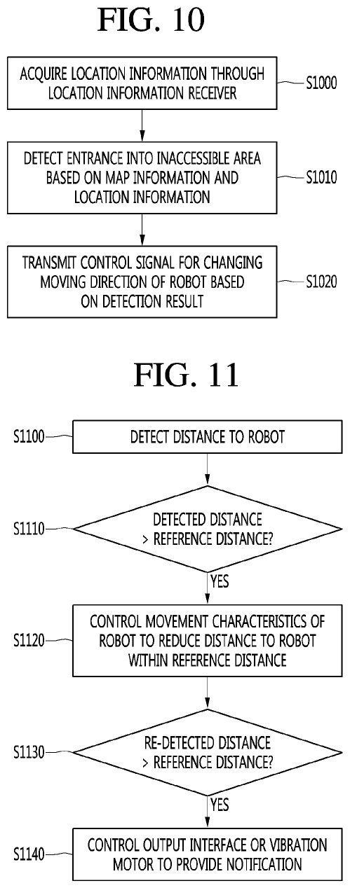

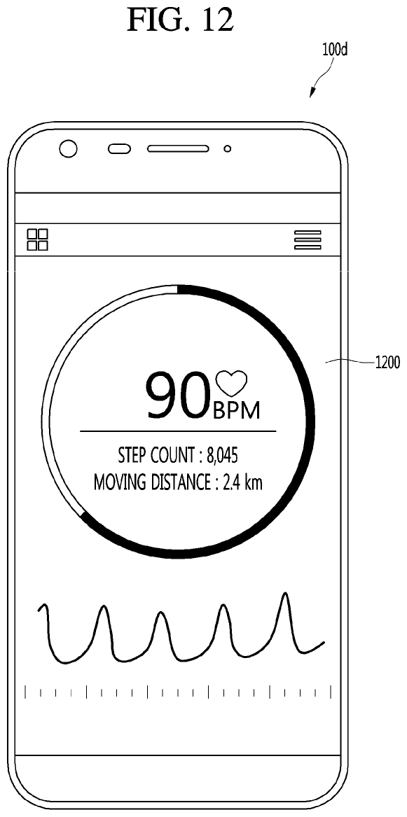

FIG. 8 is a flowchart for describing a control operation of an apparatus connected to a robot according to an embodiment. FIG. 9 illustrates an example of the user 900 walking with the robot 100a using the apparatus 600 connected to the robot 100a according to an embodiment. FIG. 10 is a flowchart for describing an operation in which the apparatus according to the embodiment controls movement characteristics of the robot. FIG. 11 is a flowchart for explaining an example of a control operation performed by the apparatus according to the embodiment, based on a distance to the robot. FIG. 12 illustrates an example in which exercise information acquired by the apparatus according to the embodiment is provided through a user terminal 100d.

Referring to FIGS. 8 and 9, the apparatus 600 can receive a request for executing a mode for generating exercise information of the user 900 (hereinafter, referred to as an exercise mode) (S800), and can check whether the apparatus 600 is connected to the robot 100a (S810).

The processor 680 can acquire the request for executing the exercise mode in various ways, and execute the exercise mode in response to the acquired request.

For example, the processor 680 can automatically execute the exercise mode when the apparatus 600 is powered on. Alternatively, the processor 680 can receive the request for executing the exercise mode through the input interface 620.

The processor 680 can check whether the robot 100a is connected to the apparatus 600 (by wire) according to the execution of the exercise mode.

According to an embodiment, the processor 680 can automatically execute the exercise mode when the wired connection between the robot 100a and the apparatus 600 is detected.

When the apparatus 600 is not connected to the robot 100a (NO in S820), the apparatus 600 can output a request for connecting to the robot 100a through the output interface 650 (S830).

When the connection to the robot 100a is detected (YES of 820), the apparatus 600 can accumulate exercise data for generating exercise information of the user 900 based on the movement of the robot 100a and the user 900 (S840).

When it is detected that the robot 100a and the apparatus 600 are connected through the robot connection portion 603 and 604, the processor 680 can acquire and accumulate (e.g., store in the memory 670) exercise data according to an exercise (for example, walking, jogging or the like) of the user 900 by using the location information receiver 618, the biometric information sensor 646, the pedometer 648, and the like.

In detail, the processor 680 can calculate a moving distance of the user 900 according to a change in the location information of the location information receiver 618. According to an embodiment, the location information can be provided from the robot 100a. In this case, the processor 680 can calculate the moving distance according to the change in the location information provided from the robot 100a.

Alternatively, the processor 680 can acquire the biometric information of the user through the biometric information sensor 646 to acquire data, such as heart rate, pulse characteristics, water content, oxygen saturation, and the like, during the exercise of the user.

Alternatively, the processor 680 can accumulate the step count of the user by using the pedometer 648.

Meanwhile, the apparatus 600 can control the movement characteristics of the robot 100a during the movement of the robot 100a and the user (S850).

The processor 680 can control the movement characteristics (moving direction and/or moving speed) of the robot 100a based on a variety of information acquired through the sensor 640 or the like.

For example, when the heart rate of the user acquired through the biometric information sensor 646 exceeds a reference heart rate, the processor 680 can determine that the moving speed of the robot 100a is too fast. Therefore, the processor 680 can generate a control signal for reducing the moving speed of the robot 100a and transmit the generated control signal to the robot 100a.

According to an embodiment, the processor 680 can receive a request for adjusting the movement characteristics of the robot 100a from the user through the input interface 620, and control the moving direction and/or the moving speed of the robot 100a in response to the received request.

Alternatively, the processor 680 can change the moving direction of the robot 100a when the robot 100a enters an inaccessible area preset or based on map information.

In this regard, referring to FIG. 10, the apparatus 600 can acquire location information through the location information receiver 618 (S1000), and can detect that the robot 100a and/or the user 900 enters the inaccessible area, based on the map information and the location information (S1010).

The processor 680 can periodically or continuously acquire the location information through the location information receiver 618 during the movement of the robot 100a and the user 900. According to an embodiment, the processor 680 can acquire the location information of the robot 100a by obtaining the location information acquired from the location information receiver of the robot 100a through the wired communication interface 616.

The processor 680 can detect whether the robot 100a and/or the user 900 has entered or intends to enter the inaccessible area in the map information, based on the acquired location information and map information. For example, when the location information corresponds to a predetermined location in the inaccessible area, or when the location information corresponds to a location within a predetermined distance from the inaccessible area, the processor 680 can detect that the robot 100a and/or the user 900 have entered or intends to enter the inaccessible area. The map information can be previously stored in the memory 670, or can be provided from the server or the like through the communication interface 610.

The apparatus 600 can transmit, to the robot 100a, a control signal for changing the moving direction of the robot 100a, based on the detection result (S1020).

When the robot 100a and/or the user 900 has entered or intends to enter the inaccessible area, the processor 680 can generate a control signal for changing the moving direction of the robot 100a, such that the robot 100a and the user 900 are spaced apart from the inaccessible area by a predetermined distance or more. The processor 680 can transmit the generated control signal to the robot 100a through the wired communication interface 616 or alternately through the short range wireless communication interface 614. The processor 180 of the robot 100a can change the moving direction of the robot 100a by controlling the driving of at least one motor included in the leg driver 162 based on the received control signal.

FIG. 8 is described again.

According to an embodiment, the apparatus 600 can control the movement characteristics of the robot 100a based on the distance to the robot 100a (S850).

For example, when the distance between the robot 100a and the apparatus 600, which is acquired through the distance sensor 642 or the short range wireless communication interface 614, is reduced to less than a predetermined minimum distance, the processor 680 can determine that the moving speed of the robot 100a is slow. Therefore, the processor 680 can generate a control signal for increasing the moving speed of the robot 100a and transmit the generated control signal to the robot 100a.

For example, when the distance between the robot 100a and the apparatus 600 exceeds a predetermined reference distance, the processor 680 can change the moving speed or the moving direction of the robot 100a.

In this regard, referring to FIG. 11, the apparatus 600 can detect a distance to the robot 100a (S1100).

For example, the processor 680 can detect the distance between the robot 100a and the apparatus 600 by using the distance sensor 642 or the short range wireless communication interface 614.

Alternatively, the processor 680 can detect whether the distance between the robot 100a and the apparatus 600 exceeds a maximum distance, based on the sensing value of the leash tension sensor 644.

When the detected distance exceeds the reference distance (YES in S1110), the apparatus 600 can control the movement characteristics of the robot 100a, such that the distance to the robot 100a decreases within the reference distance (S1120).

When the detected distance exceeds the reference distance, the processor 680 can generate a control signal for reducing the moving speed of the robot 100a or changing the moving direction of the robot 100a, and transmit the generated control signal to the robot 100a.

Alternatively, when the sensing value of the leash tension sensor 644 exceeds the reference sensing value, the processor 680 can detect that the distance between the robot 100a and the apparatus 600 exceeds the maximum distance. Therefore, the processor 680 can generate a control signal for reducing the moving speed of the robot 100a or changing the moving direction of the robot 100a, and transmit the generated control signal to the robot 100a.

The apparatus 600 can re-detect the distance to the robot 100a after a predetermined time elapses.

When the re-detected distance still exceeds the reference distance (YES in S1130), the robot 100a can control the output interface 650 and/or the vibration motor 660 to provide a notification to the user (S1140).

When the distance between the robot 100a and the apparatus 600 still exceeds the reference distance although the control signal is transmitted to the robot 100a in operation S1120, the processor 680 can detect that an abnormal situation has occurred in the robot 100a. For example, the abnormal situation can correspond to a communication failure between the robot 100a and the apparatus 600, a situation in which the robot 100a fails or cannot move, or a situation in which the robot 100a is robbed by another person.

Therefore, the processor 680 can guide the user to take appropriate measures by providing the user with the notification of the abnormal situation through the output interface 650 or the vibration motor 660.

FIG. 8 is described again.

When the apparatus 600 receives a request for ending the exercise mode (S860), the apparatus 600 can generate exercise information based on the exercise data accumulated in operation S840 (S870). The apparatus 600 can store the generated exercise information in the memory 670 or transmit the generated exercise information to an external device (a server, a mobile terminal, etc.) through the communication interface 610 (S880).

The processor 680 can receive the request for ending the exercise mode from the user through the input interface 620 and end the exercise mode. Alternatively, the processor 680 can end the exercise mode when it is detected that the robot connection portions 603 and 604 are separated from the robot 100a in a state in which the distance between the robot 100a and the apparatus 600 is less than a predetermined distance.

When the exercise mode is ended, the processor 680 can generate exercise information based on exercise data acquired and accumulated during the exercise mode (S870).

For example, the exercise information can include a variety of information, such as a moving distance, a step count, and biometric information acquired during exercise.

The processor 680 can store the generated exercise information in the memory 670 or transmit the generated exercise information to an external device, such as a server or a user mobile terminal, through the communication interface 610 (S880).

Referring to the diagram of FIG. 12, the user mobile terminal 100d can output a screen 1200 displaying exercise information received from the apparatus 600 or the server. For example, the exercise information output on the screen 1200 can include information such as a moving distance, a step count, or an average heart rate.

That is, according to the embodiments illustrated in FIGS. 8 to 12, the robot 100a and the apparatus 600 connected thereto are implemented to allow the user to perform an exercise (walking, jogging or the like) with the robot 100a, thereby improving the utilization of the robot 100a.

In addition, the apparatus 600 can automatically acquire exercise data, such as biometric information or step count, during the user's exercise and provide exercise information based on the acquired data, thereby assisting the user's effective health management.

In addition, the apparatus is connected to the robot 100a to control the movement characteristics of the robot 100a according to various situations, thereby improving the safety of the user and the robot during exercise and enabling efficient exercise.

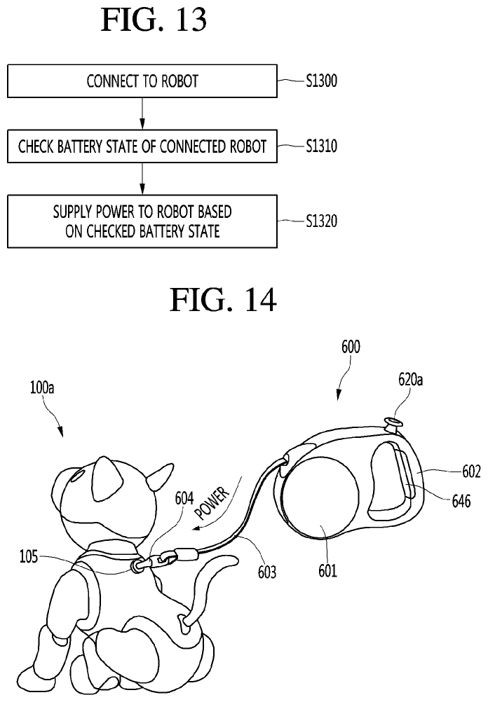

FIG. 13 is a flowchart for describing an operation in which the apparatus according to the embodiment supplies power to the robot based on a battery state of the robot. FIG. 14 illustrates an example relating to the operation of the apparatus illustrated in FIG. 13.

Referring to FIGS. 13 and 14, when the apparatus 600 is connected to the robot 100a (S1300), the apparatus 600 can check the battery state of the robot 100a (S1310).

When the processor 680 detects that the robot 100a and the apparatus 600 are connected through the robot connection portions 603 and 604, the processor 680 can acquire information (for example, remaining battery level information) related to the battery state from the robot 100a through the communication interface 610.

The apparatus 600 can supply power for charging the battery to the robot 100a based on the checked battery state (S1320).

When the remaining battery level of the robot 100a is less than a reference level, the processor 680 can control the power transmission interface 694 to supply power to the battery of the robot 100a. Therefore, the power stored in the battery 692 of the apparatus 600 can be supplied to the battery of the robot 100a through the cable 603, so that the battery of the robot 100a can be charged.

That is, when the robot 100a is driven in the outside due to the exercise of the user, the robot 100a is charged through the apparatus 600 connected to the robot 100a even if the remaining battery level is low, thereby securing sufficient usage time of the robot 100a.

According to the present embodiment(s), the robot and the apparatus connected thereto can be implemented to enable the user to perform an exercise (walking, jogging or the like) with the robot, thereby improving the utilization of the robot.

In addition, the apparatus according to the present embodiment(s) can automatically acquire exercise data, such as biometric information or step count during the user's exercise, and provide exercise information based on the acquired data, thereby assisting the user's effective health management.

In addition, the apparatus according to the present embodiment(s) is connected to the robot to control the movement characteristics of the robot according to various situations, thereby improving the safety of the user and the robot during the outside activities such as exercise and enabling efficient exercise.

In addition, according to the present embodiment(s), when the robot is driven in the outside due to the user's exercise, the robot can be charged through the apparatus connected to the robot even if the remaining battery level is insufficient, thereby ensuring sufficient usage time of the robot.

The above description is merely illustrative of the technical idea of the present disclosure, and various modifications and changes can be made thereto by those skilled in the art without departing from the essential characteristics of the present disclosure.

Therefore, the embodiments of the present disclosure are not intended to limit the technical spirit of the present disclosure but to illustrate the technical idea of the present disclosure, and the technical spirit of the present disclosure is not limited by these embodiments.

The scope of protection of the present disclosure should be interpreted by the appending claims, and all technical ideas within the scope of equivalents should be construed as falling within the scope of the present disclosure.

What is claimed is:

1. An apparatus comprising:
a communication interface configured to connect the apparatus to a robot;
a location information receiver configured to receive location information of the apparatus;
at least one sensor comprising a biometric information sensor configured to acquire biometric information of a user; and
a processor configured to:
generate exercise information of the user based on at least one of the location information of the apparatus, the biometric information of the user acquired through the biometric information sensor of the apparatus, or step count information acquired through a pedometer of the apparatus,
generate a control signal for controlling at least one of a moving direction or a moving speed of the robot, based on the location information of the apparatus or information acquired through the at least one sensor, and
control the communication interface to transmit the generated control signal to the robot.

2. The apparatus according to claim 1, wherein the biometric information sensor is configured to contact a part of the user's body to acquire the biometric information,
the biometric information includes at least one of heart rate, pulse characteristics, body temperature, water content, and oxygen saturation of the user, and
the exercise information includes at least one of a moving distance, a step count, and the acquired biometric information.

3. The apparatus according to claim 1, wherein the processor is further configured to:
detect that the location information corresponds to a location within a first predetermined distance from an inaccessible area according to map information, the map information being acquired from a memory of the apparatus or the communication interface, and the location information of the apparatus, and wherein the control signal is for changing the moving direction of the robot to position the robot apart from the inaccessible area by a second predetermined distance or more.

4. The apparatus according to claim 1, wherein the biometric information sensor is configured to detect the user's heart rate, and
wherein when the user's heart rate detected by the biometric information sensor is higher than a reference heart rate the control signal is for reducing the moving speed of the robot.

5. The apparatus according to claim 1, wherein the at least one sensor further comprises a distance sensor configured to detect a distance between the robot and the apparatus, and
wherein when the detected distance is shorter than a reference distance, the control signal is for increasing the moving speed of the robot.

6. The apparatus according to claim 1, wherein the at least one sensor further comprises a distance sensor configured to detect a distance between the robot and the apparatus, and
wherein when the detected distance is longer than a reference distance, the control signal is for reducing the moving speed of the robot or changing the moving direction of the robot.

7. The apparatus according to claim 6, wherein the apparatus includes at least one of a display, a speaker, a light source, and a vibration motor, and
wherein the processor is further configured to:
re-detect the distance between the robot and the apparatus after a predetermined time elapses from a time point when the control signal is transmitted, and
when the re-detected distance is longer than the reference distance, output a notification through the at least one of the display, the speaker, the light source and the vibration motor.

8. The apparatus according to claim 1, further comprising a cable connected to the robot,
wherein the at least one sensor further comprises a tension sensor configured to detect a tension of the cable, and
when a sensing value of the tension sensor is greater than a reference sensing value, the control signal is for reducing the moving speed of the robot or changing the moving direction of the robot.

9. The apparatus according to claim 1, further comprising an input interface configured to receive an adjustment request for adjusting the moving speed or the moving direction of the robot,
wherein the control signal is for controlling the moving speed or the moving direction of the robot according to the received adjustment request.

10. The apparatus according to claim 1, further comprising:
a cable connected to the robot, the cable including a power cable; and
a battery,
wherein the processor is further configured to:
acquire battery level information of a battery of the robot through the communication interface; and
transfer power from the battery of the apparatus to the battery of the robot through the power cable, based on the acquired battery level information.

11. The apparatus according to claim 1, further comprising a memory,
wherein the processor is further configured to store the exercise information in the memory of the apparatus, or control the communication interface to transmit the exercise information to a server or to a mobile terminal of the user.

12. A control method of an apparatus including a memory; a pedometer; and at least one sensor, the at least one sensor including a biometric information sensor, the control method comprising:
detecting a connection of the apparatus to a robot;
when an exercise mode is started, acquiring and storing, in the memory of the apparatus, exercise data including at least one of a location of the apparatus, biometric information of a user acquired through the biometric information sensor of the apparatus, or a step count acquired through the pedometer of the apparatus;
generating a control signal for controlling at least one of a moving direction or a moving speed of the robot, based on location information of the apparatus or information acquired through the at least one sensor;
transmitting the generated control signal to the robot; and
when the exercise mode ends, generating exercise information of the user based on the acquired and stored exercise data.

13. The control method according to claim 12, wherein the biometric information includes at least one of the user's heart rate, pulse characteristics, body temperature, water content, and oxygen saturation, and
the exercise information includes at least one of a moving distance, a step count, and the acquired biometric information.

14. The control method according to claim 12, wherein the generating of the control signal comprises detecting that the location information of the apparatus corresponds to a location within a first predetermined distance from an inaccessible area according to map information, and
wherein the generating of the control signal is for changing the moving direction of the robot to position the robot apart from the inaccessible area by a second predetermined distance or more.

15. The control method according to claim 12, wherein the biometric information sensor is configured to measure the user's heart rate, and
wherein when the user's heart rate detected by the biometric information sensor is higher than a reference heart rate, the generating of the control signal is for reducing the moving speed of the robot.

16. The control method according to claim 12, wherein the apparatus further comprises a distance sensor for detecting a distance between the robot and the apparatus,
wherein the method further comprises detecting the distance between the robot and the apparatus using the distance sensor, and
wherein when the detected distance is shorter than a reference distance, the generating of the control signal is for increasing the moving speed of the robot.

17. The control method according to claim 16,
when the detected distance is longer than the reference distance, the generating of the control signal is for reducing the moving speed of the robot or changing the moving direction of the robot.

18. The control method according to claim 17, wherein the apparatus further includes at least one of a display, a speaker, a light source, and a vibration motor, and
wherein the control method further comprises:
re-detecting the distance between the robot and the apparatus using the distance sensor, after a predetermined time elapses from a time point when the control signal is transmitted; and
when the re-detected distance is longer than the reference distance, outputting a notification through the at least one of the display, the speaker, the light source and the vibration motor.

19. The control method according to claim 12, wherein the apparatus comprises a cable connected to the robot,
wherein the at least one sensor further comprises a tension sensor for detecting a tension of the cable, and
wherein the generating of the control signal comprises, when a sensing value of the tension sensor is greater than a reference sensing value, the generating of the control signal is for reducing the moving speed of the robot or changing the moving direction of the robot.

20. A robot system comprising:
the apparatus according to claim 1; and
the robot connected to the apparatus.
